(19) United States
(12) Patent Application Publication
(10) Pub. No.: US 2023/0372331 A1
(43) Pub. Date: Nov. 23, 2023

(54) PHARMACEUTICAL COMPOSITION

(71) Applicant: ANXO PHARMACEUTICAL CO., LTD., Taipei City (TW)

(72) Inventors: Chia-Yu SU, Taipei City (TW); Hua-Jing JHAN, Taipei City (TW); I Hsiang LIU, Taipei City (TW); Chiung Hui HUNG, Taipei City (TW); Tse-Hsien CHEN, Taipei City (TW); Chi-Heng JIAN, Taipei City (TW)

(73) Assignee: ANXO PHARMACEUTICAL CO., LTD., Taipei City (TW)

(21) Appl. No.: 18/198,488

(22) Filed: May 17, 2023

Related U.S. Application Data
(60) Provisional application No. 63/343,143, filed on May 18, 2022.

(51) Int. Cl. A61K 31/495; A61K 47/34; A61K 9/08

(52) U.S. Cl. CPC A61K 31/495; A61K 47/34; A61K 9/08

(57) ABSTRACT

A pharmaceutical composition is provided. The pharmaceutical composition comprises an active pharmaceutical ingredient (API), a polymer and a solvent. The API comprises cariprazine, a pharmaceutically acceptable salt of cariprazine, or a mixture thereof. The polymer comprises poly(lactic acid) (PLA), poly(lactide-co-glycolide) (PLGA), poly(glycolic acid) (PGA), or a combination thereof. A method for preparing the pharmaceutical composition is also provided.

### FIELD OF THE INVENTION

The disclosure relates to a pharmaceutical composition, and relates to a flowable pharmaceutical composition comprising cariprazine for in-situ forming long-acting implants.

### DESCRIPTION OF THE RELATED ART

Cariprazine is a third-generation, orally active, and potent antipsychotic drug. It acts as dopamine D3-preferring D3/D2 receptor partial agonist and serotonin 5-HT1A receptor partial agonist with two major active metabolites, desmethyl-cariprazine (DCAR) and didesmethyl-cariprazine (DDCAR). Cariprazine (Vraylar®) was approved for the treatment of schizophrenia and bipolar disorder by the Food and Drug Administration (FDA) in 2015 and the European Medicines Agency (EMA) in 2017.

Currently, there are some disadvantages when administrating cariprazine for schizophrenia treatment with once-daily oral immediate release capsules. Schizophrenic patients usually exhibit poor adherence to treatment regimens. A therapy that includes a once-daily oral dosage can lead to irregular or inconsistent treatment, thereby risking the recurrence of psychotic episodes and crisis.

Therefore, antipsychotic drugs have evolved in order to offer better control of schizophrenia. The treatment without the need of caregivers to pay attention to daily administration and a more stable plasma concentration level in the patient after taking antipsychotic drugs are both desirable.

### BRIEF SUMMARY OF THE INVENTION

In accordance with one embodiment of the disclosure, a pharmaceutical composition is provided. The pharmaceutical composition comprises an active pharmaceutical ingredient (API), a polymer and a solvent. The API comprises cariprazine, a pharmaceutically acceptable salt of cariprazine, or a mixture thereof. The polymer comprises poly(lactic acid) (PLA), poly(lactide-co-glycolide) (PLGA), poly(glycolic acid) (PGA), or a combination thereof.

In some embodiments, the pharmaceutically acceptable salt of cariprazine comprises cariprazine HCl. In some embodiments, in the mixture of cariprazine and the pharmaceutically acceptable salt of cariprazine, the weight ratio of cariprazine to cariprazine HCl is from 1:99 to 99:1. In some embodiments, in the mixture of cariprazine and the pharmaceutically acceptable salt of cariprazine, the weight ratio of cariprazine to cariprazine HCl is from 1:3 to 3:1 or 1:1 to 1:3. In some embodiments, in the mixture of cariprazine and the pharmaceutically acceptable salt of cariprazine, the weight ratio of cariprazine to cariprazine HCl is from 1:75 to 75:1 or 1:50 to 50:1 or 1:25 to 25:1 or 1:10 to 10:1 or 1:5 to 5:1. In some embodiments, the weight ratio of the API in the pharmaceutical composition is from 1% to 30%.

In some embodiments, in PLGA, the ratio of lactide to glycolide is from 48:52 to 100:0. In some embodiments, the polymer comprises more than one PLGA with various ratios of lactide to glycolide. In some embodiments, the concentration of the polymer in the pharmaceutical composition is from 150 mg/ml to 1000 mg/ml. In some embodiments, the concentration of the polymer in the pharmaceutical composition is from 340 mg/ml to 560 mg/ml. In some embodiments, the viscosity of the polymer is from 0.1 dl/g to 0.7 dl/g.

In some embodiments, the solvent comprises water miscible solvent or partially water miscible solvent. In some embodiments, the water miscible solvent comprises dimethyl-sulfoxide (DMSO), N-methyl pyrrolidone (NMP), or a combination thereof. In some embodiments, the partially water miscible solvent comprises benzyl alcohol (BA).

In some embodiments, the weight ratio of the API to the polymer is from 1:0.8 to 1:40. In some embodiments, the weight ratio of the API to the polymer is from 1:0.9 to 1:39.6 or 1:0.9 to 1:30 or 1:0.9 to 1:20 or 1:0.9 to 1:10 or 1:0.9 to 1:5 or 1:0.9 to 1:3. In some embodiments, the weight ratio of the polymer to the solvent is from 1:0.5 to 1:4. In some embodiments, the weight ratio of the polymer to the solvent is from 1:0.5 to 1:3.5 or 1:0.5 to 1:3 or 1:0.5 to 1:2.5 or 1:0.5 to 1:2 or 1:0.5 to 1:1.5. In some embodiments, the weight ratio among the API, the polymer and the solvent is from 1:0.8:0.4 to 1:40:160. In some embodiments, the weight ratio among the API, the polymer and the solvent is from 1:0.8:0.6 to 1:30:110 or 1:0.8:0.8 to 1:20:60 or 1:0.8:1.0 to 1:10:10 or 1:0.8:1.2 to 1:3:5 or 1:0.9:1.35 to 1:3:4.5.

In some embodiments, the release percentage of the pharmaceutical composition is lower than 30% in the first 24 hours. In some embodiments, the release of the pharmaceutical composition is sustained for more than 14 days. In some embodiments, the pharmaceutical composition is stored at the temperature of 4° C. to 25° C. for use.

In accordance with one embodiment of the disclosure, a method for preparing a pharmaceutical composition is provided. The preparation method comprises the following steps. An active pharmaceutical ingredient (API) is dispersed in a solvent to form a solution. The API comprises cariprazine, a pharmaceutically acceptable salt of cariprazine, or a mixture thereof. A polymer is dissolved in the solution under continuous stirring. The polymer comprises poly(lactic acid) (PLA), poly(lactide-co-glycolide) (PLGA), poly(glycolic acid) (PGA), or a combination thereof.

A detailed description is given in the following embodiments with reference to the accompanying drawings.

### BRIEF DESCRIPTION OF THE DRAWINGS

The invention can be more fully understood by reading the subsequent detailed description and examples with references made to the accompanying drawings, wherein:

FIG. 1 shows release profiles of pharmaceutical compositions with different solvent in accordance with one embodiment of the disclosure;

FIG. 2 shows release profiles of pharmaceutical compositions with polymers of different inherent viscosities in accordance with one embodiment of the disclosure;

FIG. 3 shows release profiles of pharmaceutical compositions with different lactide (L)/glycolide (G) ratios in polymers in accordance with one embodiment of the disclosure;

FIG. 4 shows release profiles of pharmaceutical compositions with different weight ratios of PLGA polymer in accordance with one embodiment of the disclosure;

FIG. 5 shows release profiles of pharmaceutical compositions with different end groups of PLGA polymers in accordance with one embodiment of the disclosure;

FIG. 6 shows release profiles of pharmaceutical compositions with different ratios of polymer to solvent in accordance with one embodiment of the disclosure;

FIG. 7 shows release profiles of pharmaceutical compositions with different weight ratios of drug in the pharmaceutical composition in accordance with one embodiment of the disclosure;

FIG. 8 shows release profiles of pharmaceutical compositions with different weight ratios of cariprazine to polymer in accordance with one embodiment of the disclosure;

FIG. 9 shows release profiles of pharmaceutical compositions with different cariprazine salt forms in accordance with one embodiment of the disclosure;

FIG. 10 shows plasma concentration-time curves of cariprazine after subcutaneous administration of pharmaceutical compositions to rats in accordance with one embodiment of the disclosure; and

FIG. 11 shows plasma concentration-time curves of cariprazine after oral administration of Vraylar® to rats in accordance with one embodiment of the disclosure.

### DETAILED DESCRIPTION OF THE INVENTION

In accordance with one embodiment of the disclosure, a pharmaceutical composition is provided. The pharmaceutical composition comprises an active pharmaceutical ingredient (API), a polymer and a solvent. The API comprises cariprazine, a pharmaceutically acceptable salt of cariprazine, or a mixture thereof. The polymer comprises poly(lactic acid) (PLA), poly(lactide-co-glycolide) (PLGA), poly(glycolic acid) (PGA), or a combination thereof.

In some embodiments, the pharmaceutical composition may be an injectable pharmaceutical composition. In some embodiments, the pharmaceutical composition may be an in-situ forming implant upon injection. In some embodiments, the pharmaceutical composition may provide an extended release of API, for example, cariprazine. The depot is formed when contacts with water and physiological fluids and transforms in situ into the solid implant thereby providing an extended release of cariprazine for prolonged periods of time. Once the pharmaceutical composition contacts the aqueous body fluids, it solidifies to form a solid/semi-solid implant due to the solvent diffusing out. The drug release rate may be controlled by different polymer ratio.

In some embodiments, the pharmaceutically acceptable salt of cariprazine may comprise cariprazine HCl. In some embodiments, in the mixture of cariprazine and the pharmaceutically acceptable salt of cariprazine, the weight ratio of cariprazine to cariprazine HCl is from about 1:99 to about 99:1. In some embodiments, in the mixture of cariprazine and the pharmaceutically acceptable salt of cariprazine, the weight ratio of cariprazine to cariprazine HCl is from about 1:1 to about 1:3. In some embodiments, in the mixture of cariprazine and the pharmaceutically acceptable salt of cariprazine, the weight ratio of cariprazine to cariprazine HCl is from about 1:75 to about 75:1 or about 1:50 to about 50:1 or about 1:25 to about 25:1 or about 1:10 to about 10:1 or about 1:5 to about 5:1. In some embodiments, the weight ratio of the API in the pharmaceutical composition is from about 1% to about 30%. The weight ratio is expressed as the percentage of the weight of API in total pharmaceutical composition weight.

In some embodiments, the polymer may be a biodegradable and biocompatible controlled polymer. The polymer is degraded by body enzyme. As a result, the polymer is not toxic to patient, will not accumulate in the human body and will not induce inflammatory responses. The end groups of the polymer may be end-capped with esters or carboxylic acid. In some embodiments, in PLGA, the ratio of lactide to glycolide is from about 48:52 to about 100:0. The different monomer ratios in PLGA cause different time of degradation, for example, the higher content of glycolide units, the lower time required for degradation. In some embodiments, the polymer may comprise more than one PLGA with various ratios of lactide to glycolide. In some embodiment, lactide and glycolide in PLGA are mixed in a ratio from about 48:52 to about 100:0. In some embodiments, the concentration of the polymer in the pharmaceutical composition is from about 150 mg/ml to about 1000 mg/ml. In some embodiments, the concentration of the polymer in the pharmaceutical composition is from about 340 mg/ml to about 560 mg/ml. In some embodiments, the viscosity of the polymer is from about 0.1 dl/g to about 0.7 dl/g. In some embodiments, the viscosity of the polymer is from about 0.14 dl/g to about 0.60 dl/g. In some embodiments, the viscosity of the polymer is from about 0.14 dl/g to about 0.24 dl/g. Inherent viscosity is measured in chloroform at 25° C., at a concentration of 0.1% wt/v by viscometer. The inherent viscosity is related to polymer molecular weight. The polymers with higher molecular weight will increase the viscosity of liquid in which they dissolved. In some embodiments, the average molecular weight of the polymer is from about 4 kD to about 54 kD. In some embodiments, the average molecular weight of the polymer is from about 4 kD to about 17 kD.

Solvent is used for the injection into any living body. The needs of solvent are susceptible not to cause toxicity. Solvent is biocompatible, not to cause tissue necrosis or irritation at the injection site. For the in-situ forming implant, the solvent should diffuse quickly from the polymeric solution toward surrounding tissues when exposed to body fluids. Solvent diffusion leads to polymer precipitate, and the implant formulation effectively leads to sustained release of active ingredient encapsulated. In some embodiment, the solubility of polymer in solvent is more than 1%. In some embodiments, the solvent may comprise water miscible solvent or partially water miscible solvent. In some embodiments, the water miscible solvent may comprise dimethyl-sulfoxide (DMSO), N-methyl pyrrolidone (NMP), or a combination thereof. In some embodiments, the partially water miscible solvent may comprise benzyl alcohol (BA).

The weight ratio of API to polymer in the pharmaceutical composition may also contribute to control the initial release of API from the implant. In some embodiments, the weight ratio of the API to the polymer is from about 1:0.8 to about 1:40. In another embodiments, the weight ratio of the API to the polymer is from about 1:0.9 to about 1:39.6 or about 1:0.9 to about 1:30 or about 1:0.9 to about 1:20 or about 1:0.9 to about 1:10 or about 1:0.9 to about 1:5 or about 1:0.9 to about 1:3. In some embodiments, the weight ratio of the polymer to the solvent is from about 1:0.5 to about 1:4. In some embodiments, the weight ratio of the polymer to the solvent is from about 1:0.5 to about 1:3.5 or about 1:0.5 to about 1:3 or about 1:0.5 to about 1:2.5 or about 1:0.5 to about 1:2 or about 1:0.5 to about 1:1.5. In some embodiments, the weight ratio among the API, the polymer and the solvent is from about 1:0.8:0.4 to about 1:40:160. In some embodiments, the weight ratio among the API, the polymer and the solvent is from about 1:0.8:0.6 to about 1:30:110 or about 1:0.8:0.8 to about 1:20:60 or about 1:0.8:1.0 to about 1:10:10 or about 1:0.8:1.2 to about 1:3:5 or about 1:0.9:1.35 to about 1:3:4.5.

In some embodiment, the pharmaceutical composition may further comprise an excipient. In some embodiment, the excipient may be, for example, an antioxidant.

In some embodiments, the release percentage of the pharmaceutical composition may be lower than about 30% in the first 24 hours. In some embodiments, the release percentage of the pharmaceutical composition may be lower than about 25% in the first 24 hours. In some embodiments, the release percentage of the pharmaceutical composition may be lower than about 20% in the first 24 hours. In some embodiments, the release percentage of the pharmaceutical composition may be lower than about 15% in the first 24 hours. In some embodiments, the release percentage of the pharmaceutical composition may be lower than about 10% in the first 24 hours. In some embodiments, the sustained release of the pharmaceutical composition may be more than 14 days. In some embodiments, the sustained release of the pharmaceutical composition may be more than 20 days. In some embodiments, the sustained release of the pharmaceutical composition may be more than 25 days. In some embodiments, the sustained release of the pharmaceutical composition may be more than 28 days. In some embodiments, the sustained release of the pharmaceutical composition may be more than 30 days. In some embodiments, the sustained release of the pharmaceutical composition may be more than 1.5 months. In some embodiments, the sustained release of the pharmaceutical composition may be more than 3 months. In some embodiments, the sustained release of the pharmaceutical composition may be more than 6 months. In some embodiments, the pharmaceutical composition is stored from about 4° C. to about 25° C. for use.

In accordance with one embodiment of the disclosure, a method for preparing a pharmaceutical composition is provided. The preparation method comprises the following steps. An active pharmaceutical ingredient (API) is dispersed in a solvent to form a solution. The API comprises cariprazine, a pharmaceutically acceptable salt of cariprazine, or a mixture thereof. A polymer is dissolved in the solution under continuous stirring. The polymer comprises poly(lactic acid) (PLA), poly(lactide-co-glycolide) (PLGA), poly(glycolic acid) (PGA), or a combination thereof.

In some embodiments, another method for preparing a pharmaceutical composition is provided. The preparation method includes the following steps. A polymer is dissolved in a solvent under continuous stirring to form a solution. An active pharmaceutical ingredient (API) is dispersed in the solution.

### Example 1

Preparation of Pharmaceutical Composition and Drug Release Method

The pharmaceutical composition was prepared as follows. First, cariprazine or its pharmaceutically acceptable salt was dispersed in solvent selected from dimethyl-sulfoxide (DMSO), N-methyl-pyrrolidone (NMP) or benzyl alcohol (BA) to form a suspension solution. Subsequently, PLGA polymer selected from 502H, 503H, 653H, 752H and 752S was dissolved in the suspension solution under continuous stirring for 10 min. The mixture of solution was put overnight until PLGA polymer dissolved completely.

To measure the drug release profile of cariprazine, 50 mg of pharmaceutical composition was injected into 10 mL acetate buffer in a glass vial, capped and placed inside an incubator shaker at 37° C., 100 rpm to form a medium. At predetermined time points (t=1, 2, 4, 7, 10, 14, 21 and 28 days), medium was collected to measure the release of cariprazine. At each time point, 2 mL of medium was collected and replaced with 2 mL fresh acetate buffer to form a dissolution sample. The dissolution sample was analyzed by High Performance Liquid Chromatography (HPLC) under the condition described in Table 1.

TABLE 1

| Method parameter | Description | |
|---|---|---|
| Wavelength | 215 | nm |
| Inject volume | 20 | μL |
| Flow rate | 1.0 | mL/min |
| Run time | 13 mins (RT of cariprazine: about 10 min) | |
| Mobile phase | pH 6.5 solution: ACN = 4:6 | |
| Column | YMC triart C18, 4.6*250 mm, 5 μm (YM6-3) | |
| Column temperature | 35° | C. |
| Sample temperature | 20° | C. |

| | |
|---|---|
| Diluent and Needle wash | ACN: MeOH: H₂O = 7:2:1 |

### Example 2

Study of Solvent Suitable for Using in Pharmaceutical Composition

In the present invention, solvent suitable for using in pharmaceutical composition was first to be estimated. It was known that the solubility of polymer in solvent could affect the formation of pharmaceutical composition and the release of cariprazine from the pharmaceutical composition. As a result, the solubility of polymer in solvent was estimated to find a suitable range for using in pharmaceutical composition.

In the present example, the pharmaceutical compositions were as follows.

The pharmaceutical composition comprising cariprazine was prepared by dispersing cariprazine in the water miscible solvent, partially water miscible solvent or water un-miscible solvent respectively to form a solution, and subsequently dissolving PLGA (model: 502H) polymer in the solution. Water miscible solvent such as NMP and DMSO belonged to fast phase inversion system and highly polar solvents. Partially water-miscible solvent such as benzyl alcohol (BA) belonged to slow inverting system. Water un-miscible solvent such as benzyl benzoate (BB) belonged to non-polar solvent. The pharmaceutical compositions with different solvents were named TF8-BB, TF8-BA, TF8-N and TF8-D and showed in Table 2.

TABLE 2

| Pharmaceutical | | Component amount (mg) | | |
|---|---|---|---|---|
| composition | Solvent | Cariprazine | Polymer | Solvent |
| TF8-BB | BB (Benzyl benzoate) | 64 | 174 | 262 |
| TF8-BA | BA (Benzyl alcohol) | 64 | 174 | 262 |
| TF8-N | NMP (N-methyl- | 64 | 174 | 262 |
| | pyrrolidone) | | | |
| TF8-D | DMSO (Dimethyl- | 64 | 174 | 262 |
| | sulfoxide) | | | |

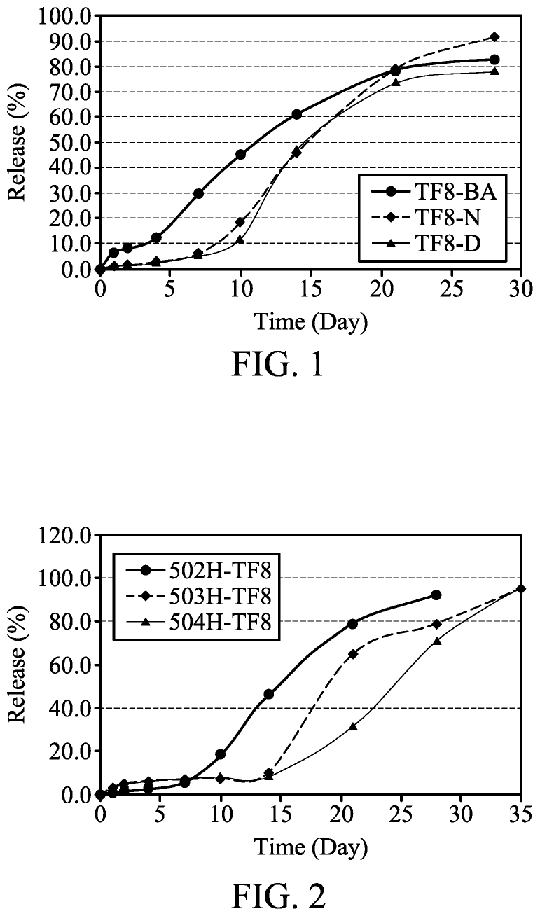

The results showed that BB solvent was not a suitable solvent for pharmaceutical compositions because PLGA polymer couldn't dissolve well in BB solvent. However, pharmaceutical composition with BA solvent (TF8-BA) formed an implant which was very soft and easy to change shape. FIG. 1 showed release profiles of pharmaceutical compositions with different solvent in accordance with this example. As shown in FIG. 1, the release profile of cariprazine released from the pharmaceutical composition with DMSO solvent (TF8-D) was similar to the pharmaceutical composition with NMP solvent (TF8-N). Besides, both of the accumulated release percentage of cariprazine released from TF8-D and TF8-N in 1 day were less than 10%. The NMP and DMSO solvent were better than BA solvent for using in pharmaceutical composition because pharmaceutical compositions TF8-N and TF8-D showed slower drug release in 14 days. It was shown that release of cariprazine was closely related to the solvent property.

### Example 3

Study of Polymers with Different Inherent Viscosities

In the present example, the pharmaceutical compositions were as follows.

The pharmaceutical compositions comprising cariprazine were prepared by dispersing cariprazine in the NMP solvent and subsequently dissolving PLGA polymer with different inherent viscosity (IV). The models of PLGA polymer were 502H, 503H, 504H and 505, wherein the 502H polymer had the lowest IV and the 505 polymer had the highest IV. The pharmaceutical compositions with different PLGA polymers were named 502H-TF8, 503H-TF8, 504H-TF8, and 505-TF8 and showed in Table 3.

TABLE 3

| | | Polymer | | | |
|---|---|---|---|---|---|
| | | Inherent | | | |
| Pharmaceutical | | Viscosity | Component amount (mg) | | |
| composition | Polymer | (IV, dl/g) | Cariprazine | Polymer | Solvent |
| 502H-TF8 | 502H | 0.16-0.24 | 64 | 174 | 262 |
| 503H-TF8 | 503H | 0.32-0.44 | 64 | 174 | 262 |
| 504H-TF8 | 504H | 0.45-0.60 | 64 | 174 | 262 |
| 505-TF8 | 505 | 0.61-0.74 | 64 | 174 | 262 |

FIG. 2 showed release profiles of pharmaceutical compositions with polymers of different inherent viscosities in accordance with the example. The results showed that the release rate of cariprazine in pharmaceutical composition with PLGA polymer which had lower IV was more quickly (pharmaceutical composition named 502H-TF8) than in pharmaceutical composition with PLGA polymer which had higher IV (pharmaceutical compositions named 503H-TF8 and 504H-TF8) and could sustain to 14 days. However, 505 polymer was not suitable for using in pharmaceutical composition because the 505 polymer couldn't dissolve in NMP solvent. The accumulated percentage of cariprazine released on day 1, day 14 and day 28 was 1.00%, 46.00% and 91.66% in pharmaceutical composition 502H-TF8; 2.94%, 10.15% and 78.78% in pharmaceutical composition 503H-TF8; 2.96%, 8.20% and 70.80% in pharmaceutical composition 504H-TF8, respectively. Compared to pharmaceutical composition 503H-TF8, NMP solvent in pharmaceutical composition 502H-TF8 containing PLGA polymer which had lower IV would cause slower diffusion of NMP solvent and lead to slower initial cariprazine release rate. However, pharmaceutical composition 502H-TF8 containing PLGA polymer which had lower IV showed increased release profile because of its degradation rate would be faster than those containing PLGA polymer which had higher IV. Besides, pharmaceutical compositions containing PLGA polymer which had lower inherent viscosity would have less solution viscosity and easy for injection.

### Example 4

Study of Different Lactide (L)/Glycolide (G) Ratio in Polymers

Poly (lactide-glycolide) (PLGA) polymers are copolymers with different ratios of lactide(L) to glycolide(G) (L/G ratio). This example investigated influence of different L/G ratio in polymers used in pharmaceutical compositions on the in vitro release of cariprazine. In the present example, the pharmaceutical compositions were as follows.

The pharmaceutical compositions comprising cariprazine were prepared by dispersing cariprazine in the NMP solvent and subsequently dissolving different models of PLGA polymers such as 502H, 653H, 752H and 202H, respectively. The pharmaceutical compositions with different ratios of lactide to glycolide in polymers were named B-502H-TF8-N, B-653H-TF8-N, B-752H-TF8-N, B-202H-TF8-N, and different weight combinations of polymer types were named B-502H/752H-TF8-N(1:1) and B-502H/752H-TF8-N(1:3) and showed in Table 4.

TABLE 4

| | | Polymer | | | |
|---|---|---|---|---|---|
| | L/G ratio | inherent | | | |
| Pharmaceutical | in PLGA | viscosity | Component amount (mg) | | |
| composition | polymer | (dl/g) | Cariprazine | Polymer | Solvent |
| B-502H-TF8-N | 50:50 | 0.16-0.24 | 64 | 174 | 262 |
| B-653H-TF8-N | 65:35 | 0.32-0.44 | 64 | 174 | 262 |
| B-752H-TF8-N | 75:25 | 0.14-0.22 | 64 | 174 | 262 |
| B-202H-TF8-N | 100:0 | 0.16-0.24 | 64 | 174 | 262 |
| B-502H/752H- | 50:50; 75:25 | 0.16-0.24 | 64 | 174 | 262 |
| TF8-N (1:1) | (weight ratio | | | | |
| | 1:1) | | | | |
| B-502H/752H- | 50:50; 75:25 | 0.16-0.24 | 64 | 174 | 262 |
| TF8-N (1:3) | (weight ratio | | | | |
| | 1:3) | | | | |

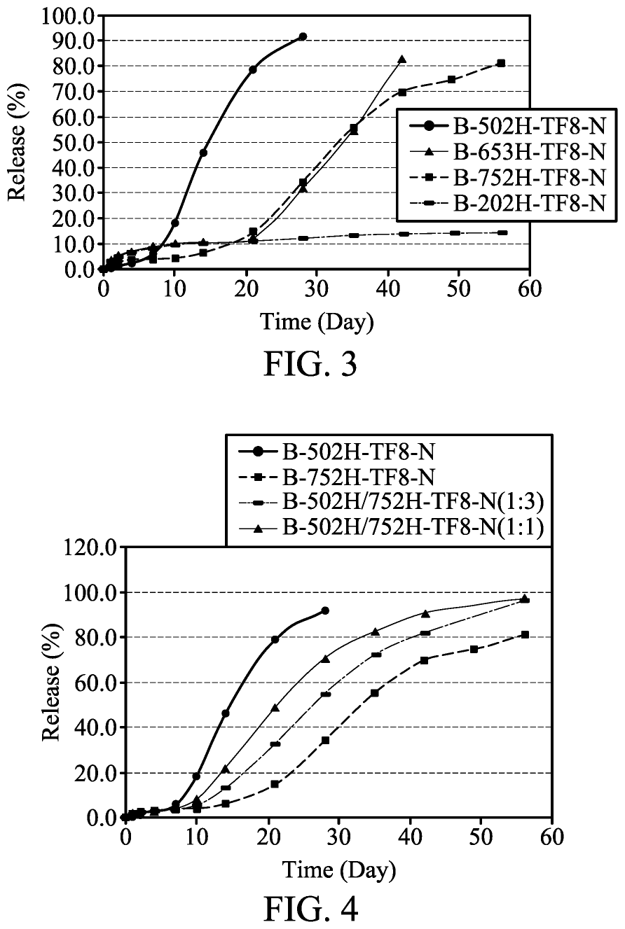

The result showed that release rate of cariprazine from the pharmaceutical compositions with higher L/G ratio was slower than those with lower L/G ratio. FIG. 3 showed release profiles of pharmaceutical compositions with different L/G ratios in polymers in accordance with the example. As shown in FIG. 3, the accumulated release percentage of pharmaceutical compositions with 502H PLGA polymer on day 1, day 14 and day 28 were 1.00%, 46.00% and 91.66%, respectively. The accumulated release percentage of pharmaceutical composition with 752H PLGA polymer on day 1, day 14 and day 28 were 1.70%, 6.50% and 34.30%, respectively. On the other group, the accumulated release percentage of pharmaceutical compositions with 653H PLGA polymer on day 1, day 14 and day 28 were 3.51%, 10.49% and 31.71%, respectively. The accumulated release percentage of pharmaceutical compositions with 202H PLGA polymer on day 1, day 14 and day 28 were 3.58%, 9.90% and 11.95%, respectively. As the result, degradation of implant formed from pharmaceutical composition with higher L/G ratio polymer was slower because of the presence of hydrophobic group in PLGA polymer, and further resulting to slower absorption and diffusion of water.

To investigate the influence of the different weight ratio of PLGA polymer combination on the release of cariprazine. The pharmaceutical compositions were prepared with the combination of 752H PLGA polymer and 502H PLGA polymer. FIG. 4 showed release profiles of pharmaceutical compositions with different weight ratios of PLGA polymer in accordance with the example. As shown in FIG. 4, the release profile of cariprazine from pharmaceutical compositions with different weight ratios of PLGA polymer combination were between pharmaceutical compositions with only 502H PLGA polymer or 752H PLGA polymer. The accumulated release percentage of cariprazine from pharmaceutical compositions B-502H-TF8-N, B-502H/752H-TF8-N(1:1), B-502H/752H-TF8-N(1:3) and B-752H-TF8-N, is over 10.0% on Day 10, 14, 14 and 21, respectively, and the accumulated release percentage of cariprazine nearly 80.0% happened on Day 28, 35, 42 and 56, respectively.

### Example 5

Study of Different End Group of PLGA Polymers

In the present example, the pharmaceutical compositions were as follows.

The pharmaceutical compositions comprising cariprazine HCl were prepared by dispersing cariprazine in the NMP solvent and subsequently dissolving PLGA polymer with different end group. The models of PLGA polymers were 752H and 752S, wherein the 752H polymer had carboxylic end group and the 752S polymer had ester end group. The pharmaceutical compositions with different PLGA polymers were named C-752H-TF8 and C-752S-TF8 and showed in Table 5.

TABLE 5

| | | Component amount (mg) | | |
|---|---|---|---|---|
| Pharmaceutical | | Cariprazine | | |
| composition | Polymer end group | HCl | Polymer | Solvent |
| C-752H-TF8 | carboxylic group | 69.1 | 172.4 | 258.5 |
| C-752S-TF8 | ester group | 69.1 | 172.4 | 258.5 |

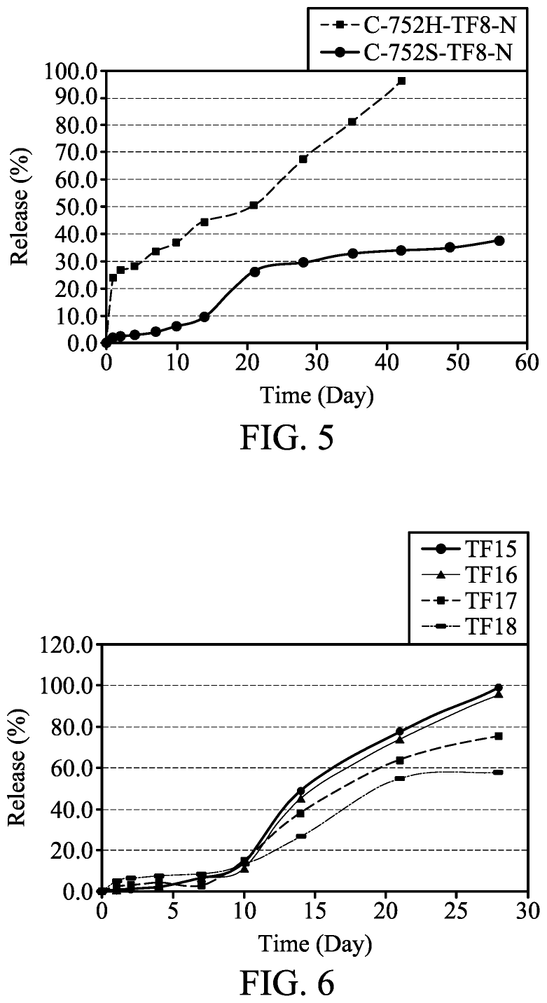

FIG. 5 showed release profiles of pharmaceutical compositions with different end groups of PLGA polymers in accordance with the example. As shown in FIG. 5, the initial drug release of cariprazine HCl from pharmaceutical compositions with PLGA polymers which had ester end group on 24 hour was below 30% and sustained to about 56 days. However, as shown in FIG. 5, pharmaceutical compositions with PLGA polymers which had carboxylic end group or ester end group could both lead to sustained release profile of cariprazine HCl.

### Example 6

Study of Different Weight Ratio of Polymer to Solvent

In the present example, the pharmaceutical compositions were as follows.

The pharmaceutical compositions comprising cariprazine were prepared by dispersing cariprazine in the NMP solvent and subsequently dissolving 502H PLGA polymer. The weight ratios of polymer to solvent were 1:0.65, 1:0.8, 1:2, 1:4, and 1:9. The pharmaceutical compositions with different weight ratios of polymer to solvent were named TF15, TF16, TF17, TF18 and TF19 and showed in Table 6.

TABLE 6

| | Component amount (mg) | | | |
|---|---|---|---|---|
| Pharmaceutical | | | | Weight ratios of |
| composition | Cariprazine | Polymer | Solvent | polymer to solvent |
| TF15 | 64 | 264 | 172 | 1:0.65 |
| TF16 | 64 | 242 | 194 | 1:0.8 |
| TF17 | 64 | 145 | 291 | 1:2 |
| TF18 | 64 | 87 | 349 | 1:4 |
| TF19 | 64 | 44 | 392 | 1:9 |

It was showed that the pharmaceutical composition TF19 with weight ratio of polymer to solvent 1:9 couldn't form an implant. Since each of the formulation of pharmaceutical composition in Table 6 is summed up to 500 mg, and which is about 0.5 ml. The result showed concentration of polymer in pharmaceutical composition to form an implant should be within 174 mg/ml to 528 mg/ml. FIG. 6 showed release profiles of pharmaceutical compositions with different ratios of polymer to solvent in accordance with the example. As shown in FIG. 6, accumulated release percentage of cariprazine released from the pharmaceutical compositions decreased with higher weight ratio of polymer to solvent. The accumulated release percentage of cariprazine on 24 hour was below 10% in all pharmaceutical compositions. Besides, it showed that pharmaceutical compositions with weight ratio of polymer to solvent range from 1:0.65 to 1:4 lead to sustained release profile of cariprazine.

### Example 7

Study of Different Weight Ratio of Cariprazine in the Pharmaceutical Compositions

In the present example, the pharmaceutical compositions were as follows.

The pharmaceutical compositions comprising cariprazine were prepared by dispersing cariprazine in the NMP solvent and subsequently dissolving PLGA polymer model 502H. The weight ratios of cariprazine in the pharmaceutical compositions were between 1-30% (w/w). The weight ratio of cariprazine was calculated by the weight of cariprazine to the weight of total pharmaceutical composition. The pharmaceutical compositions were shown in Table 7.

TABLE 7

| | Component amount (mg) | | | |
|---|---|---|---|---|
| | | | | Weight ratio |
| | | | | of Cariprazine |
| | | | | (cariprazine/total |
| Pharmaceutical | | | | pharmaceutical |
| compositions | Cariprazine | Polymer | Solvent | composition) |
| TF8 | 64 | 174 | 262 | 12.8% |
| TF13 | 64 | 2534 | 3801 | 1% |
| TF14 | 64 | 59.7 | 89.6 | 30% |

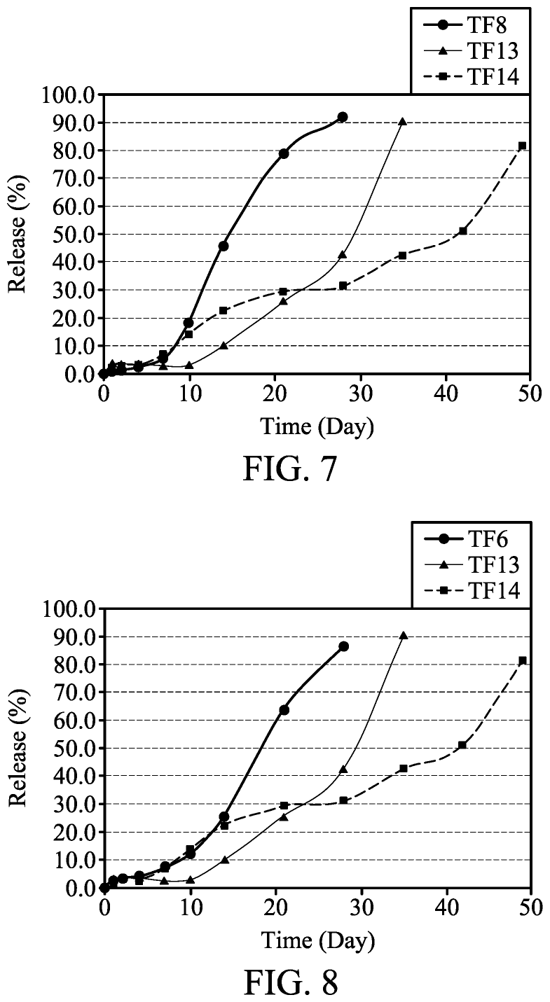

FIG. 7 showed release profiles of pharmaceutical compositions with different weight ratios of drug in the pharmaceutical composition in accordance with the example. As shown in FIG. 7, the pharmaceutical composition TF14 showed sustained release of cariprazine to about 49 days. High weight ratio of cariprazine loaded in pharmaceutical composition lead to smaller injection volume and greater patience compliance. The release of cariprazine in the first 7 days had no obvious difference in pharmaceutical compositions with different weight ratios of cariprazine loaded in the pharmaceutical composition ranging from 1% to 30%.

### Example 8

Study of Different Weight Ratios of Cariprazine to Polymer

In the present example, the pharmaceutical compositions were as follows.

The pharmaceutical compositions comprising cariprazine were prepared by dispersing cariprazine in the NMP solvent and subsequently dissolving PLGA polymer 502H. The weight ratios of cariprazine to polymer were 1:0.9 to 1:39.6. The weight ratio was calculated by the weight of cariprazine to the weight of polymer. The pharmaceutical compositions with different weight ratios of cariprazine to polymer were named TF6, TF13 and TF14 and showed in Table 8.

TABLE 8

| | Component amount (mg) | | | |
|---|---|---|---|---|
| Pharmaceutical | | | | Weight ratio of |
| composition | Cariprazine | Polymer | Solvent | cariprazine to polymer |
| TF6 | 64 | 374 | 562 | 1:5.8 |
| TF13 | 64 | 2534 | 3801 | 1:39.6 |
| TF14 | 64 | 59.7 | 89.6 | 1:0.9 |

FIG. 8 showed release profiles of pharmaceutical compositions with different weight ratios of cariprazine to polymer in accordance with the example. It was showed that the release of cariprazine from pharmaceutical composition with weight ratio of cariprazine to polymer ranging from 1:0.9 to 1:39.6 could sustain for 28 to 49 days.

### Example 9

Study of Different Cariprazine Salt Forms Used in Pharmaceutical Compositions

In the present example, the pharmaceutical compositions were as follows.

The pharmaceutical compositions were prepared by dispersing cariprazine base and HCl form in the NMP solvent and subsequently dissolving PLGA polymer model 752H, respectively. Pharmaceutical compositions with different salt forms of cariprazine were illustrated in Table 9 and were named B-752H-TF8-N, C-752H-TF8-N, B/C-752H-TF8-N (1:3) and B/C-752H-TF8-N(1:1).

TABLE 9

| | | Component amount (mg) | | | |
|---|---|---|---|---|---|
| Pharmaceutical | Cariprazine | Cariprazine | | | |
| composition | salt form | Base | HCl | Polymer | Solvent |
| B-752H-TF8-N | Base | 64 | | 174 | 262 |
| C-752H-TF8-N | HCl | | 69.1 | 172.4 | 258.5 |
| B/C-752H-TF8- | Base/HCl | 16 | 51.8 | 172.9 | 259.3 |
| N (1:3) | (weight ratio: | | | | |
| | 1:3) | | | | |
| B/C-752H-TF8- | Base/HCl | 32 | 34.6 | 173.4 | 260.1 |
| N (1:1) | (weight ratio: | | | | |
| | 1:1) | | | | |

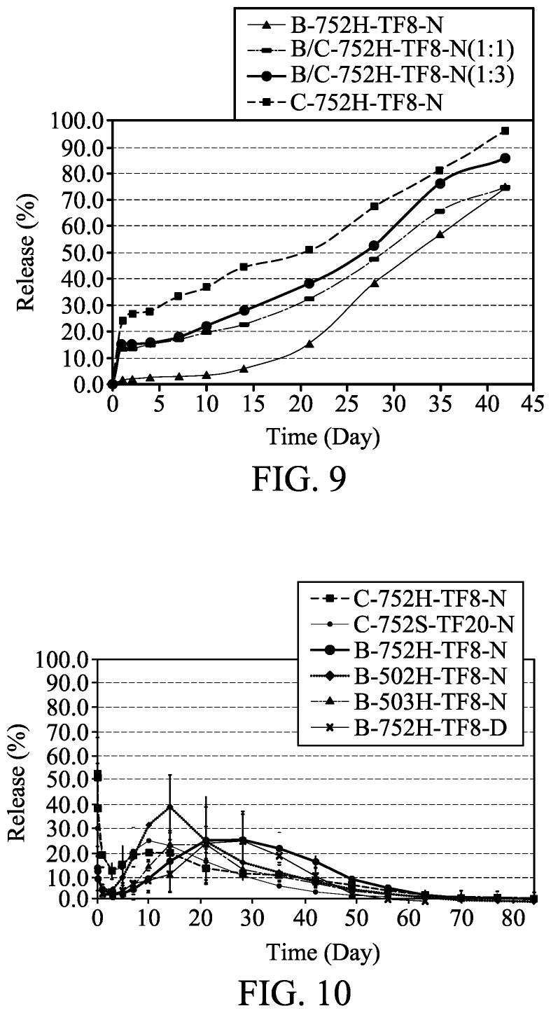

FIG. 9 showed release profiles of pharmaceutical compositions with different cariprazine salt forms in accordance with the example. As shown in FIG. 9 the accumulated release percentage of cariprazine HCl in the pharmaceutical composition was about 25% in 24 hr and showed faster release profile than pharmaceutical composition comprising cariprazine base. Different drug solubilities of cariprazine HCl and cariprazine base could be the reason to affect the release of cariprazine in the pharmaceutical compositions. Cariprazine HCl had higher solubility in acetate medium than cariprazine base. The release profile of cariprazine in pharmaceutical composition with combo cariprazine base and cariprazine HCl were between pharmaceutical compositions B-752H-TF8-N and C-752H-TF8-N.

### Example 10

Stability of Pharmaceutical Compositions

In the present example, the pharmaceutical compositions were as follows.

The pharmaceutical composition comprising cariprazine was prepared by dispersing cariprazine in the NMP solvent and subsequently dissolving PLGA polymer 502H. The pharmaceutical composition was named TF20 and showed in Table 10. The test of dissolution in this stability study was conduct by acceleration dissolution method.

TABLE 10

| Pharmaceutical | Component amount (mg) | | |
|---|---|---|---|
| composition | Cariprazine | Polymer | Solvent |
| TF20 | 60 | 176 | 264 |

Table 11 showed stability investigation of pharmaceutical composition TF20 stored at 4° C., and table 12 showed stability investigation of pharmaceutical composition TF20 stored at room temperature (25° C.). It was known that the pharmaceutical composition for injection usually stored at 4° C. However, the results showed the pharmaceutical compositions of the present invention stored at room temperature (25° C.) remained steady. Both pharmaceutical compositions stored at 4° C. and 25° C. respectively meet quality specification. That is, pharmaceutical compositions of the present invention could be stored at room temperature and lead to reduce of cost of manufacture.

TABLE 11

| Item | Specification | Initial | 1M | 2M | 4M |
|---|---|---|---|---|---|
| Dissolution | 1 D ≤25% | 2.4% | 1.3% | 1.7% | 1.8% |
| | 5.5 D: 44% to 74% | 54.7% | 49.4% | 48.3% | 50.0% |
| | 10 D: ≥80% | 90.5% | 86.0% | 85.2% | 85.6% |
| Assay | 90% to 110% | 99.5% | 96.9% | 103.1% | 98.8% |

TABLE 12

| Item | Specification | Initial | 1M | 3M |
|---|---|---|---|---|
| Dissolution | 1 D ≤25% | 2.4% | 5.9% | 15.4% |
| | 5.5 D: 44% to 74% | 54.7% | 58.8% | 65.5% |
| | 10 D: ≥80% | 90.5% | 93.1% | 95.6% |
| Assay | 90% to 110% | 99.5% | 99.8% | 99.1% |

### Example 11

Rat Pharmacokinetics Studies of Pharmaceutical Compositions

The pharmaceutical compositions were injected subcutaneous to 3 to 5 Wistar Rats with average weight of 250 g. About 75 mg/kg cariprazine was injected subcutaneous in the dorsal thoracic using a syringe with a 20G needle. The pharmaceutical compositions were shown in Table 13. Drug plasma concentration after subcutaneous administration was evaluated. After injection, plasma concentration were obtained at 0, 0.15, 0.21, 1 day, 3 days, 5 days, 7 days, 10 days, 14 days, 21 days, 28 days, 35 days, 42 days, 49 days and periodically up to 56 days, 63, 70, 77, and 84 days, respectively and shown in FIG. 10.

TABLE 13

| | | | Component amount (mg) | | | |
|---|---|---|---|---|---|---|
| Pharmaceutical | | | Cariprazine | | | |
| composition | Polymer | solvent | Base | HCl | Polymer | Solvent |
| C-752H-TF8-N | 752H | NMP | — | 69.1 | 172.4 | 258.5 |
| C-752S-TF20-N | 752S | NMP | — | 64.8 | 174.1 | 261.1 |
| B-752H-TF8-N | 752H | NMP | 64 | — | 174 | 262 |
| B-502H-TF8-N | 502H | NMP | 64 | — | 174 | 262 |
| B-503H-TF8-N | 503H | NMP | 64 | — | 174 | 262 |
| B-752H-TF8-D | 752H | DMSO | 64 | — | 174 | 262 |

FIG. 10 showed plasma concentration-time curves of cariprazine after subcutaneous administration of pharmaceutical compositions to rats in accordance with one embodiment of the example. The pharmacokinetics (PK) parameter was calculated by WinNonlin software and shown in Table 14. The PK profile was found to be related to the L/G ratio. The higher L/G ratio in the pharmaceutical composition lead to the slower release rate and lower maximum plasma concentration (Cₘₐₓ). However, different inherent viscosities of polymer showed similar Tₘₐₓ (time to reach Cₘₐₓ). In addition, pharmaceutical composition with different solvent (e.g. NMP or DMSO) showed similar release profile of cariprazine. Different solvent used in the pharmaceutical composition like NMP and DMSO was not the main reason to affect the release of cariprazine. Different end group polymers used in the pharmaceutical composition like 752H and 752S were not a main reason to affect the drug absorption in vivo. The results showed pharmaceutical compositions of the present invention had no burst release in 24 hours. The release of cariprazine from the pharmaceutical compositions could be sustained for more than 14 days. More preferably, the release of cariprazine from the pharmaceutical compositions could be sustained for more than 30 days, even more than 3 months.

TABLE 14

| | T_{1/2} | Cₘₐₓ | Tₘₐₓ | AUCₐₗₗ |
|---|---|---|---|---|
| Formulation | (day) | (ng/mL) | (day) | (day*ng/mL) |
| C-752H-TF8-N | 14.5 | 52.5 | 0.13 | 788.7 |
| C-752S-TF20-N | 14.1 | 59.2 | 0.13 | 661.7 |
| B-752H-TF8-N | 10.2 | 25.2 | 28 | 933.2 |
| B-502H-TF8-N | 7.0 | 39.1 | 14 | 925.8 |
| B-503H-TF8-N | 37.0 | 24.0 | 14 | 727.1 |
| B-752H-TF8-D | 4.4 | 24.9 | 28 | 720.6 |

In addition, six rats orally administered 5-day repeated oral dose 0.27 mg/kg of Vraylar® as control group. Blood samples were collected in tubes from the tail vein at 0.16, 0.5, 1, 2, 3, 5, 7, 10, and 24 hours on the first & fifth day and 1 and 3 hr on the second to fourth day after oral administration. After collection, all blood samples were immediately centrifuged to obtain plasma, and then stored at −80° C. before analysis. Plasma cariprazine extraction was performed by protein precipitation and analyzed by LC-MS/MS.

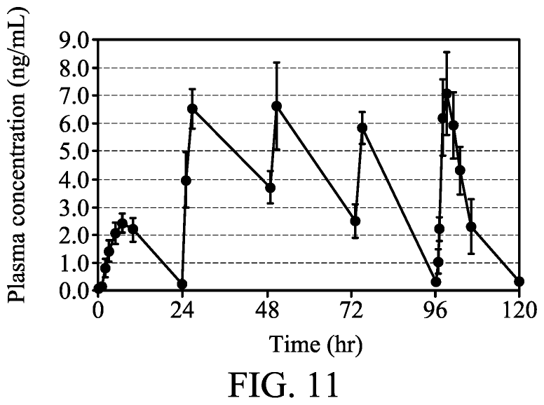

FIG. 11 showed plasma concentration-time curves of cariprazine after oral administration of Vraylar® to rats in accordance with one embodiment of the example. The pharmacokinetics (PK) parameter was calculated by WinNonlin software and shown in Table 15. As shown in Table 15, the half-life (T_{1/2}) of Vraylar® was 4.5 hr, wherein half-life (T_{1/2}) of the pharmaceutical compositions of the present invention was from 4.4 day to 37.0 day.

TABLE 15

| | Day 1 | Day 5 |
|---|---|---|
| T_{1/2} (hr) | 4.73 ± 0.83 | 4.45 ± 0.72 |
| Cₘₐₓ (ng/mL) | 2.54 ± 0.37 | 7.08 ± 1.49 |
| Tₘₐₓ (hr) | 7.17 ± 1.60 | 3.00 ± 0.00 |
| AUCₐₗₗ | 33.32 ± 5.46 | 63.06 ± 11.92 |
| hr*ng/mL) | | |

The result showed that the pharmaceutical composition of present invention could provide longer sustain release than the oral dosage form of cariprazine. The results also showed that the pharmaceutical composition of the present invention could lead to less fluctuation of cariprazine concentration in plasma, and cariprazine released from the pharmaceutical composition could be sustained for at least 84 days.

While the invention has been described by way of example and in terms of the preferred embodiments, it should be understood that the invention is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A pharmaceutical composition, comprising:
an active pharmaceutical ingredient (API) comprising cariprazine, a pharmaceutically acceptable salt of cariprazine, or a mixture thereof;
a polymer comprising poly(lactic acid) (PLA), poly(lactide-co-glycolide) (PLGA), poly(glycolic acid) (PGA), or a combination thereof; and
a solvent.

2. The pharmaceutical composition as claimed in claim 1, wherein the pharmaceutically acceptable salt of cariprazine comprises cariprazine HCl.

3. The pharmaceutical composition as claimed in claim 2, wherein, in the mixture of cariprazine and the pharmaceutically acceptable salt of cariprazine, cariprazine and cariprazine HCl have a weight ratio from 1:99 to 99:1.

4. The pharmaceutical composition as claimed in claim 2, wherein, in the mixture of cariprazine and the pharmaceutically acceptable salt of cariprazine, cariprazine and cariprazine HCl have a weight ratio from 1:1 to 1:3.

5. The pharmaceutical composition as claimed in claim 1, wherein the API has a weight ratio in the pharmaceutical composition from 1% to 30%.

6. The pharmaceutical composition as claimed in claim 1, wherein, in PLGA, lactide and glycolide have a ratio from 48:52 to 100:0.

7. The pharmaceutical composition as claimed in claim 6, wherein the polymer comprises more than one PLGA with various ratios of lactide to glycolide.

8. The pharmaceutical composition as claimed in claim 1, wherein the polymer has a concentration in the pharmaceutical composition from 150 mg/ml to 1000 mg/ml.

9. The pharmaceutical composition as claimed in claim 1, wherein the polymer has a concentration in the pharmaceutical composition from 340 mg/ml to 560 mg/ml.

10. The pharmaceutical composition as claimed in claim 1, wherein the polymer has a viscosity from 0.1 dl/g to 0.7 dl/g.

11. The pharmaceutical composition as claimed in claim 2, wherein the solvent comprises water miscible solvent or partially water miscible solvent.

12. The pharmaceutical composition as claimed in claim 11, wherein the water miscible solvent comprises dimethyl-sulfoxide (DMSO), N-methyl pyrrolidone (NMP), or a combination thereof.

13. The pharmaceutical composition as claimed in claim 11, wherein the partially water miscible solvent comprises benzyl alcohol (BA).

14. The pharmaceutical composition as claimed in claim 1, wherein the API and the polymer have a weight ratio from 1:0.8-1:40.

15. The pharmaceutical composition as claimed in claim 1, wherein the polymer and the solvent have a weight ratio from 1:0.5 to 1:4.

16. The pharmaceutical composition as claimed in claim 1, wherein the API, the polymer and the solvent have a weight ratio from 1:0.8:0.4 to 1:40:160.

17. The pharmaceutical composition as claimed in claim 3, wherein the pharmaceutical composition has a release percentage lower than 30% in the first 24 hours.

18. The pharmaceutical composition as claimed in claim 1, wherein the pharmaceutical composition has a sustained release of more than 14 days.

19. The pharmaceutical composition as claimed in claim 6, wherein the pharmaceutical composition is stored at a temperature of 4° C. to 25° C. for use.

20. A method for preparing a pharmaceutical composition, comprising:
dispersing an active pharmaceutical ingredient (API) in a solvent to form a solution, wherein the API comprises cariprazine, a pharmaceutically acceptable salt of cariprazine, or a mixture thereof; and
dissolving a polymer in the solution under continuous stirring, wherein the polymer comprises poly(lactic acid) (PLA), poly(lactide-co-glycolide) (PLGA), poly(glycolic acid) (PGA), or a combination thereof.
